(19) United States
(12) Patent Application Publication
(10) Pub. No.: US 2021/0008411 A1
(43) Pub. Date: Jan. 14, 2021

(54) LUNG CLEARING AND BODY BUILDING MACHINE

(71) Applicant: Likai Fu, Dongfang (CN)

(72) Inventor: Likai Fu, Dongfang (CN)

(21) Appl. No.: 16/940,412

(22) Filed: Jul. 28, 2020

(51) Int. Cl. A63B 23/18; A61M 16/00; A63B 24/00

(52) U.S. Cl. CPC A63B 23/18; A61M 16/0006; A63B 24/0062; A63B 2220/833; A63B 2225/50; A63B 2230/40; A61M 2016/0036

(57) ABSTRACT

The present invention discloses a lung clearing and body building machine which mainly comprises a blowing pipe, a support frame, a ball body and a cover body. The blowing pipe comprises an air inlet end and an air outlet end. The air outlet end is provided with the support frame. The ball body is limited in the support frame. The air outlet end is buckled with the cover body, and a seal ring is arranged at a joint. Absorbent wool is arranged inside the cover body. In the present invention, by adding the absorbent wool in the cover body and injecting medicine into the absorbent wool, a use effect is enhanced. The present invention integrates the functions of prevention, treatment and health care, has convenient use, good atomization effect and no stimulation, and can monitor the use situation in real time.

### TECHNICAL FIELD

The present invention relates to the technical field of lung clearing and body building machines, and more particularly relates to a lung clearing and body building machine.

### BACKGROUND

### Technology

At present, with the improvement of living standards and the progress of science and technology, an existing method of sputum suction is called sputum suction with a nasal catheter, in which a sputum suction catheter of a connected sputum suction apparatus is inserted into the nasal cavity, a patient is stimulated to cough and the sputum that is difficult to cough out by the patient is sucked out. The method of sputum suction is applied to older patients with weak expectoration. The method greatly stimulates the nasal cavity because the stimulated nasal mucosa is very easy to bleed and the patient subjected to sputum suction is especially uncomfortable due to the hardness of the sputum suction catheter and the pressure of the sputum suction apparatus. Moreover, the method has the problems of high apparatus cost and inconvenient carrying. Furthermore, the ventilators currently introduced on the market need electricity to allow one catheter to generate positive pressure and the other catheter to generate negative pressure. Two respiratory catheters generate positive pressure and negative pressure to help the patients with poor cardiorespiratory function and breathing difficulty. Such ventilators are extremely inconvenient, have poor use effect, and also have certain harm to the human body.

Therefore, the problem to be urgently solved by those skilled in the art is how to provide a ventilator with good medical effect and convenient use.

### SUMMARY

In view of this, the present invention provides a lung clearing and body building machine with good medical effect, convenient use, no stimulation and easy use.

To achieve the above purpose, the present invention provides the following technical solution:

A lung, clearing and body building machine mainly comprises: a blowing pipe, a support frame, a ball body and a cover body, wherein the blowing pipe comprises an air inlet end and an air outlet end; the air outlet end is provided with the support frame; the ball body is limited in the support frame; the air outlet end is buckled with the cover body, and a seal ring is arranged at a joint; absorbent wool is arranged inside the cover body; a miniature air flow sensor, a signal transmitting device and a power supply assembly are arranged inside the blowing pipe, wherein the miniature air flow sensor is connected with the signal transmitting device; the power supply assembly supplies power for the miniature air flow sensor and the signal transmitting device; the signal transmitting device is composed of a microprocessor, an encoder, a filter and a signal transmitting module; and the data detected by the miniature air flow sensor is processed, encoded, and then transmitted to a terminal through the signal transmitting module.

Preferably, in the above lung clearing and body building machine, the bottom of the cover body is provided with clamping grooves; the air outlet end of the blowing pipe is provided with buckles around the circumference; and the cover body and the blowing pipe are fixedly connected with each other through the buckles and the clamping grooves.

Preferably, in the above lung clearing and body building machine, the blowing pipe is provided with a protecting sleeve.

Preferably, in the above lung clearing and body building machine, the support frame is of a conical structure.

Preferably, in the above lung clearing and body building machine, first vent holes are formed in the top of the cover body.

Preferably, in the above lung clearing and body building machine, the air inlet end and the air outlet end adopt an integrated structure, and the air inlet end and the air outlet end form a certain angle, so that the blowing pipe is in a hook shape.

Preferably, in the above lung clearing and body building machine, corresponding detecting information can be acquired on the terminal through a mobile phone APP.

Preferably, in the above lung clearing and body building machine, the detecting information comprises use time, use frequency and blowing frequency.

It can be known from the above technical solution that compared with the prior art, the present invention provides a lung clearing and body building machine. In the present invention, by adding the absorbent wool in the cover body and injecting medicine into the absorbent wool, a use effect is enhanced. By adding the signal transmitting device and the miniature air flow sensor in the blowing pipe, the use situation is further monitored in real time so that people better understand the use situation. In the present invention, by adding the absorbent wool in the cover body and injecting the medicine into the absorbent wool, the use effect is enhanced. The present invention integrates the functions of prevention, treatment and health care, has convenient use, good atomization effect and no stimulation, and can monitor the use situation in real time.

### DESCRIPTION OF DRAWINGS

To more clearly describe the technical solution in the embodiments of the present invention or in the prior art, the drawings required to be used in the description of the embodiments or the prior art will be simply presented below. Apparently, the drawings in the following description are merely the embodiments of the present invention, and for those ordinary skilled in the art, other drawings can also be obtained according to the provided drawings without contributing creative labor.

FIG. 1 is a structural schematic diagram of the present invention.

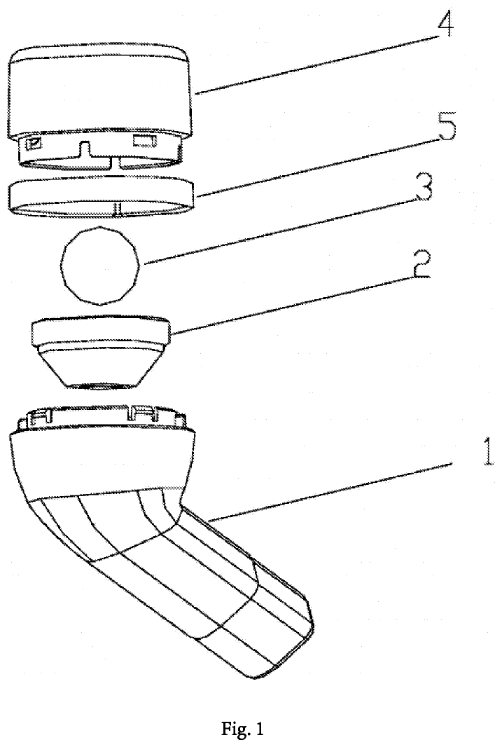

FIG. 2 is an exploded structural schematic diagram of the present invention.

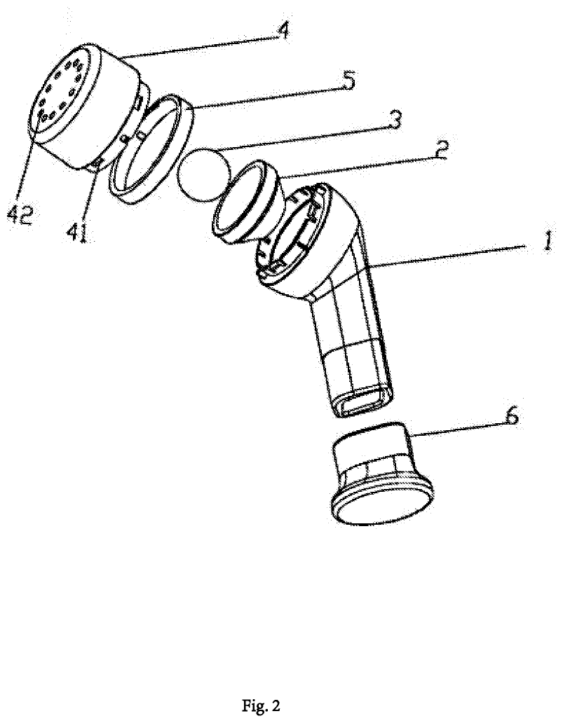

### DETAILED DESCRIPTION

The technical solution in the embodiments of the present invention will be clearly and fully described below in combination with the drawings in the embodiments of the present invention. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

Embodiments of the present invention disclose a lung clearing and body building machine.

The lung clearing and body building machine mainly comprises: a blowing pipe 1, a support frame 2, a ball body 3 and a cover body 4, wherein the blowing pipe 1 comprises an air inlet end 11 and an air outlet end 12; the air outlet end 12 is provided with the support frame 2; the ball body 3 is limited in the support frame 2: the air outlet end 12 is buckled with the cover body 4, and a seal ring 5 is arranged at a joint; absorbent wool is arranged inside the cover body 4; a miniature air flow sensor, a signal transmitting device and a power supply assembly are arranged inside the blowing pipe 1, wherein the miniature air flow sensor is connected with the signal transmitting device; the power supply assembly supplies power for the miniature air flow sensor and the signal transmitting device; the signal transmitting device is composed of a microprocessor, an encoder, a filter and a signal transmitting module; and the data detected by the miniature air flow sensor is processed, encoded, and then transmitted to a terminal through the signal transmitting module.

To further optimize the above technical solution, the bottom of the cover body 4 is provided with clamping grooves 41; the air outlet end 12 of the blowing pipe 1 is provided with buckles around the circumference; and the cover, body and the blowing pipe are fixedly connected with each other through the buckles and the clamping grooves 41. The seal ring 5 seals the joint of the buckles and the clamping grooves 41 to avoid air leakage.

To further optimize the above technical solution, the blowing pipe 1 is provided with a protecting sleeve 6.

To further optimize the above technical solution, the support frame 2 is of a conical structure to accelerate the flow of airflows.

To further optimize the above technical solution, first vent holes 42 are formed in the top of the cover body 4 so that air which returns to the mouth is as fresh as possible.

To further optimize the above technical solution, the air inlet end 11 and the air outlet end 12 adopt an integrated structure, and the air inlet end 11 and the air outlet end 12 form a certain angle, so that the blowing pipe 1 is in a hook shape.

To further optimize the above technical solution, corresponding detecting information can be acquired on the terminal through a mobile phone APP.

To further optimize the above technical solution, the detecting information comprises use time, use frequency and blowing frequency.

To further optimize the above technical solution, the working principle of the present invention is as follows: firstly, the medicine is injected into the absorbent wool inside the cover body 4; a user holds the blowing pipe 1 of the lung clearing and body building machine with hands and blows the air inlet end 11 with the mouth; and the airflow enters the air outlet end 12 along an air inlet pipe, and then enters the support frame 2. Under the action of the airflow force, the ball body 3 is blown, and the airflow returns when touching the cover body 4; because the airflow returns through the absorbent wool with the medicine, the airflow at this time is attached with medicinal particles; the airflow enters the air outlet end 12 and returns to the air inlet end 11, thereby entering the mouth of a human body, entering the throat of the human body and entering the lungs of the human body; thus, the airflow in the lungs of the human body rotates back and forth; the backflow of the airflow and the blown gas collide with each other in the lungs to generate vibration, so as to loosen the alveoli, enable dust and other foreign objects attached to the alveoli to fall off and then spit out the foreign objects through cough, thereby achieving the effect of lung clearing. Meanwhile, when the user blows with the lung clearing and body building machine of embodiments of the present invention, the backflow of the airflow is accelerated so that the cardiorespiratory function is exercised, thereby enhancing the blood circulation of the body and increasing the vital capacity to achieve the effect of body building.

To further optimize the above technical solution, the miniature air flow sensor and the signal transmitting device are arranged in the blowing pipe 1; and through the cooperation between the miniature air flow sensor and the signal transmitting device, the use data of the user is transmitted to the terminal in real time and viewed through the mobile phone APP.

To further optimize the above technical solution, the technologies of the signal transmitting device, the miniature air flow sensor and the viewing of the data in the terminal through the mobile phone APP are the existing known technologies. For example, a weather station detects the air quality through an air quality sensor, and transmits the detected data to the terminal for viewing the data through the corresponding mobile phone APP. They have the same information transmission mode, so the corresponding technical features will not be repeated.

Each embodiment in the description is described in a progressive way. The difference of each embodiment from each other is the focus of explanation. The same and similar parts among all of the embodiments can be referred to each other. For the device disclosed by the embodiments, because the device corresponds to a method disclosed by the embodiments, the device is simply described. Refer to the description of the method part for the related part.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

1. A lung clearing and body building machine, mainly comprising: a blowing pipe, a support frame, a ball body and a cover body, wherein the blowing pipe comprises an air inlet end and an air outlet end; the air outlet end is provided with the support frame; the ball body is limited in the support frame; the air outlet end is buckled with the cover body, and a seal ring is arranged at a joint; absorbent wool is arranged inside the cover body; a miniature air flow sensor, a signal transmitting device and a power supply assembly are arranged inside the blowing pipe, wherein the miniature air flow sensor is connected with the signal transmitting device; the power supply assembly supplies power for the miniature air flow sensor and the signal transmitting device; the signal transmitting device is composed, of a microprocessor, an encoder, a filter and a signal transmitting module; and the data detected by the miniature air flow sensor is processed, encoded, and then transmitted to a terminal through the signal transmitting module.

2. The lung clearing and body building machine according to claim 1, wherein the bottom of the cover body is provided with clamping grooves; the air outlet end of the blowing pipe is provided with buckles around the circumference; and the cover body and the blowing pipe are fixedly connected with each other through the buckles and the clamping grooves.

3. The lung clearing and body building machine according to claim 2, wherein the blowing pipe is provided with a protecting sleeve.

4. The lung clearing and body building machine according to claim 1, wherein the support frame is of a conical structure.

5. The lung clearing and body building machine according to claim 2, wherein first vent holes are formed in the top of the cover body.

6. The lung clearing and body building machine according to claim 1, wherein the air inlet end and the air outlet end adopt an integrated structure, and the air inlet end and the air outlet end form a certain angle, so that the blowing pipe is in a hook shape.

7. The lung clearing and body building machine according to claim 1, wherein corresponding detecting information can be acquired on the terminal through a mobile phone APP.

8. The lung clearing and body building machine according to claim 7, wherein the detecting information comprises use time, use frequency and blowing frequency.
